(19) United States
(12) Patent Application Publication
(10) Pub. No.: US 2010/0247607 A1
(43) Pub. Date: Sep. 30, 2010

(54) IMPLANTABLE FORMULATIONS OF BISPHOSPHONIC ACIDS

(75) Inventor: Paul Ashton, Newton, MA (US)

Correspondence Address: ROPES & GRAY LLP, PATENT DOCKETING 39/41, ONE INTERNATIONAL PLACE, BOSTON, MA, 02110-2624, US

(73) Assignee: pSivida US, Inc., Watertown, MA (US)

(21) Appl. No.: 12/732,385

(22) Filed: Mar. 26, 2010

Related U.S. Application Data
(60) Provisional application No. 61/211,298, filed on Mar. 26, 2009.

(51) Int. Cl. A61F 2/00; A61P 35/04; A61P 19/08; A61P 19/10; C07F 9/6506; A61K 31/675; C07F 9/38; A61K 31/663; C07F 9/58; C07F 3/06

(52) U.S. Cl. 424/426; 548/112; 514/94; 562/13; 514/108; 546/22; 514/89; 556/18; 424/423

(57) ABSTRACT

The invention relates to devices, methods and formulation for subcutaneous administration of a bisphosphonate. In such a device, a drug core, comprising a bisphosphonate, is disposed in a tube. The devices may be administered to a patient in need of subcutaneously wherein the release of the bisphosphonate is desired to provide sustained release of a therapeutically effective dose of the bisphosphonate.

### RELATED APPLICATION

This application claims the benefit of priority to U.S. Provisional Application Ser. No. 61/211,298, filed Mar. 26, 2009. The specification of the foregoing application is hereby incorporated by reference in its entirety.

### FIELD OF THE INVENTION

The present invention relates to novel drug delivery devices for administration of a bisphosphonate, and methods of use and manufacture thereof.

### BACKGROUND OF THE INVENTION

A variety of disorders in humans and other mammals involve or are associated with abnormal bone resorption. Such disorders include, but are not limited to, osteoporosis, glucocorticoid-induced osteoporosis, Paget's disease, abnormally increased bone turnover, periodontitis, tooth loss, bone fractures, rheumatoid arthritis, osteoarthritis, periprosthetic osteolysis, osteogenesis imperfecta, metastatic bone disease, hypercalcemia of malignancy, and multiple myeloma. One of the most common of these disorders is osteoporosis, which in its most frequent manifestation occurs in postmenopausal women. Osteoporosis is a systemic skeletal disease characterized by a low bone mass and microarchitectural deterioration of bone tissue, with a consequent increase in bone fragility and susceptibility to fracture. Osteoporotic fractures are a major cause of morbidity and mortality in the elderly population. As many as 50% of women and a third of men will experience an osteoporotic fracture. A large segment of the older population already has low bone density and a high risk of fractures. There is a significant need to both prevent and treat osteoporosis and other conditions associated with bone resorption.

Metastatic bone disease involves tumor-induced skeletal metastases, which commonly result from breast cancer, prostate cancer, lung cancer, renal cancer, thyroid cancer, and multiple myeloma. The prevalence of bone metastases in patients with these cancers may be as high as 60-85%. Patients with these diseases that have bone dominant or bone-only metastases frequently have prolonged survival usually associated with clinical morbidity. The most frequent clinical manifestations of bone metastases are pain, pathological fracture, immobility, nerve root or spinal cord compression, hypercalcemia, and compromised hematopoiesis. The scope of metastatic bone disease is highlighted by the fact that on any given day, approximately 4 million people worldwide suffer from cancer pain and that at least 40-50% of all cancer pain is due to skeletal metastases. Current therapy for treating metastatic bone disease has limited efficacy with only a modest reduction in skeletal events associated with the disorder. Furthermore, many agents have undesirable side effects. There remains a need for therapeutic options with improved efficacy and reduced adverse events.

One of the recently investigated approaches for prevention of bone loss and bone resorption is the administration of bisphosphonates. Bisphosphonates were found to prevent bone resorption leading to reduction of bone fractures, especially the fractures of spine and hip. Recent studies have demonstrated that these compounds prevent the loss of bone and enhance bone density in the postmenopausal female population and in patients with Paget's disease of bone (Journal of Clinical Endocrinology and Metabolism, 82(1):265-274 (1997); Journal of Bone and Mineral Research, 12(10):1700-1707 (1997); American Journal of Medicine, 106(5):513-520 (1997); Journal of Clinical Endocrinology and Metabolism, 83(2):396-402 (1998)). Cumulatively, the above references show that bisphosphonates prevent a breakdown of bone, strengthen bone, increase a bone mass and markedly reduce fractures.

Bisphosphonates were additionally investigated for treatment of cancer, as described, for example, in New England Journal of Medicine, 335:1785-1791 (1996), which reports a decreased frequency of skeletal events in patients with multiple myeloma involving bone and breast cancer with osteolytic metastases following a treatment with bisphosphonates. Clinical trials described in New England Journal of Medicine, 339:398-400 (1998) have shown that adjunctive treatment with bisphosphonates reduces the incidence and number of new bone and visceral metastases in women with high risk, primary breast cancer.

Numerous bisphosphonates are now available for therapeutic use; however, their administration and delivery remains problematic.

A preferred mode of drug delivery is by oral administration. However, this mode of administration of bisphosphonates is limited by the low gastrointestinal absorption and by overall low gastrointestinal tolerability of bisphosphonates. Gastrointestinal absorption of the bisphosphonates is very poor and, typically, only about 1% or less of the administered dose is bioavailable.

Additionally, a significant number of women treated with oral bisphosphonates were reported to develop irritation of esophageal mucosa, esophageal reflux and esophagitis (Digestive Diseases and Sciences, 43 (9):1998-2002 (1998); Digestive Diseases and Sciences, 43(5):1009-1015 (1998)). To lessen these undesirable adverse reactions, the oral administration of bisphosphonates generally follows a very strict regimen which can be inconvenient or even burdensome for patients.

In view of the difficulties associated with administration of bisphosphonates, improved delivery techniques are needed.

### SUMMARY OF THE INVENTION

The present invention relates to novel drug delivery devices for administration of a bisphosphonate, and methods of use and manufacture thereof.

In one aspect, the present invention provides a drug delivery device, wherein the device comprises a drug core comprising a low-solubility salt of a bisphosphonate.

In certain embodiments, the drug delivery device provides sustained release of a therapeutically effective dose of a bisphosphonate over a prolonged period, e.g., over a period of at least 1 day, at least 2 days, or even at least 1 week, at least 1 month, or at least 6 months, or at least 1 year. In an exemplary embodiment, the drug delivery device provides sustained release of a therapeutically effective dose of a bisphosphonate for six to twelve months upon administration.

In certain embodiments, the drug core of the device is disposed in a tube that has first and second ends. In preferred embodiments, the tube is sized and formed of a material so that the tube is dimensionally stable and capable of supporting its own weight.

In certain embodiments, the tube is permeable to passage of the bisphosphonate and/or salt thereof, while in others, the tube is impermeable to passage of the bisphosphonate and/or the salt. In certain embodiments, the tube has first and second ends, and preferably at least one of the first or second ends is open or covered by a layer that is permeable to passage of the bisphosphonate and/or the salt.

In certain embodiments, the tube further comprises a cap abutting the first and/or second ends of the tube, wherein each cap is independently permeable or impermeable to the passage of the bisphosphonate and/or the salt. In certain embodiments, the device comprises a cap at each end, and at least one cap is permeable to passage of the bisphosphonate and/or the salt.

In certain embodiments, the cap comprises at least one of the following polymers: (poly(vinyl acetate) (PVAC), poly(caprolactone) (PCL), polyethylene glycol (PEG), poly(dl-lactide-co-glycolide) (PLGA), ethylene vinyl acetate polymer (EVA), poly(vinyl alcohol) (PVA), poly(lactic acid) (PLA), poly(glycolic acid) (PGA), polyalkyl cyanoacralate, polyurethane, or nylon, or a copolymer thereof. In certain embodiments, the cap is bioerodible.

In certain embodiments, the drug delivery devices of the invention further comprises an outer layer that covers at least a portion of the tube and/or its ends, wherein the outer layer may be permeable or impermeable to the passage of the bisphosphonate and/or the salt. In certain embodiments, the outer layer covers at least one end and is permeable to passage of the bisphosphonate and/or the salt. In certain embodiments, each end is covered with an outer layer that may be permeable or impermeable to the passage of the bisphosphonate and/or the salt. In certain embodiments, one end is covered with an outer layer that is impermeable to the passage of the bisphosphonate and/or the salt and the other end is coating with a layer that is permeable to the passage of the bisphosphonate and/or the salt; in other embodiments, each end is covered with an outer layer that is permeable to the passage of the bisphosphonate and/or the salt.

In certain embodiments, the outer layer comprises at least one of the following polymers: poly(vinyl acetate) (PVAC), poly(caprolactone) (PCL), polyethylene glycol (PEG), poly(dl-lactide-co-glycolide) (PLGA), ethylene vinyl acetate polymer (EVA), poly(vinyl alcohol) (PVA), poly(lactic acid) (PLA), poly(glycolic acid) (PGA), polyalkyl cyanoacrylate, polyurethane, or nylon, or a copolymer thereof. In certain embodiments, the outer layer is bioerodible.

In certain embodiments, the tube comprises a polymer, a metal, or silicon. In certain embodiments, the tube is bioerodible.

In certain embodiments, the tube comprises at least one of the following impermeable bioerodible polymers: ethylene vinyl acetate polymer (EVA), polyalkyl cyanoacrylate, polyurethane, nylon, or a copolymer thereof.

In certain embodiments, the bisphosphonate salt is a salt of a bisphosphonic acid with a divalent cation, such as a divalent metal cation. In certain embodiments, the divalent metal cation is selected from calcium, magnesium, manganese, and zinc. In an exemplary embodiment, the divalent metal cation is magnesium.

In certain embodiments, the bisphosphonate is selected from alendronate, clodronate, etidronate, pamidronate, tiludronate, ibandronate, zoledronate, olpadronate, risedronate, incadronate, minodronate, and neridronate. In certain embodiments, the bisphosphonate is selected from ibandronate, zoledronate, and risedronate. In an exemplary embodiment, the bisphosphonate is zoledronate.

Accordingly, in certain embodiments, the invention relates to a salt of ibandronate selected from calcium, zinc and benzathine.

In certain embodiments, the inventions relates to a salt of zoledronate selected from magnesium, calcium and zinc.

In certain embodiments, the invention relates to a salt of risedronate selected from magnesium and calcium.

In certain embodiments, the drug delivery device of the invention comprises from about 10 mg to about 20 mg of a bisphosphonate.

In another aspect, the present invention provides a method for administering a bisphosphonate to a patient, comprising subcutaneously implanting the drug delivery device of the invention, e.g., a drug device comprising a drug core comprising a low-solubility salt of a bisphosphonate. In an exemplary embodiment, upon administration, the drug delivery device provides sustained release of a therapeutically effective dose of a bisphosphonate for six to twelve months.

In certain embodiments, the patient is suffering from osteoporosis, Paget's disease, or bone metastases.

### BRIEF DESCRIPTION OF THE DRAWINGS

Throughout the figures, like reference numerals designate identical or corresponding elements.

FIG. 1 is an enlarged cross-sectional illustration of one embodiment of a drug delivery device in accordance with the present invention.

FIG. 2 is an enlarged cross-sectional illustration of the embodiment illustrated in FIG. 1 with additional optional parts.

FIG. 3 is an enlarged cross-sectional illustration of the embodiment illustrated in FIG. 1 with additional optional parts.

FIG. 4 is an enlarged cross-sectional illustration of the embodiment illustrated in FIG. 1 with additional optional parts.

FIG. 5 is an enlarged cross-sectional illustration of a second embodiment of a drug delivery device in accordance with the present invention.

FIG. 6 is an enlarged cross-sectional illustration of a third embodiment of a drug delivery device in accordance with the present invention.

FIG. 7 is an enlarged cross-sectional illustration of a fourth embodiment of a drug delivery device in accordance with the present invention.

FIG. 8 is a cross-sectional illustration of the embodiment illustrated in FIG. 6, taken at line 8-8.

FIG. 9 schematically illustrates an embodiment of a method in accordance with the present invention of fabricating a drug delivery device.

FIG. 10 shows the release profile (measured as μg/day) of the 0.9 mm magnesium zoledronate implants.

FIG. 11 shows the release profile (measured as μg/day) of the 2.0 mm magnesium zoledronate implants.

FIG. 12 shows the release profile of an exemplary lipophilic/low-solubility agent, spironolactone.

### DETAILED DESCRIPTION OF THE INVENTION

### 1. Overview

The invention generally relates to novel devices and methods for administration of a bisphosphonate to a patient. In particular, the invention discloses novel drug delivery formulations and devices that provide sustained release of a therapeutically effective dose of a bisphosphonate over a prolonged period (e.g., for six to twelve months). Compared with oral or intravenous administration of bisphosphonates, the drug delivery devices of the invention may enhance the absorption or bioavailability of a bisphosphonate, provide more convenient administration, and/or provide a sustained release of bisphosphonate within the therapeutic range at a relatively constant level over a prolonged period of time.

The invention further relates to methods of using the novel drug delivery devices for treatment or prevention of bone resorption, loss of bone mass and/or strength, osteoporosis, Paget's disease, nonmetastatic neoplastic disease, bone metastases, and other bone or skeletal diseases.

### 2. Bisphosphonates

Bisphosphonates are a class of drugs for use in a variety of diseases of bone and calcium metabolism. Bisphosphonates are synthetic analogs of pyrophosphates characterized by a phosphorus-carbon-phosphorus backbone that renders them resistant to hydrolysis. The properties of the bisphosphonates vary based on different substitutions at the carbon atom of the phosphorus-carbon-phosphorus backbone.

Bisphosphonates include, but are not limited to, alendronate, clodronate, etidronate, pamidronate, tiludronate, ibandronate, zoledronate, olpadronate, risedronate, incadronate, minodronate, and neridronate.

a. Representative Bisphosphonates

Zoledronate (zoledronic acid) is marketed by Novartis under the trade names Zometa® and Reclast®.

Alendronate is commercially available from Merck & Co., Inc., Rahway, N.J. as alendronate sodium under the product name Fosamax®.

Clodronate is commercially available from Roche Diagnostics GmbH (Mannheim, Germany).

Etidronate is commercially available from Procter & Gamble Pharmaceuticals (Cincinnati, Ohio) under the product name Didronel®.

Pamidronate (aminohydroxypropylidine bisphosphonate, APD) is commercially available from Novartis, Basel, Switzerland under the product name Aredia®.

Tiludronate (tiludronic acid disodium salt) is commercially available from Sanofi, France under the product name Skelid®.

Ibandronate is commercially available from Roche Diagnostics GmbH (Mannheim, Germany) under the product name Boniva®.

Neridronate (6-Amino-1-hydroxyhexylidene bisphosphonic acid) is a third generation amino-bisphosphonate.

Risedronate is commercially available from Procter & Gamble Pharmaceuticals (Cincinnati, Ohio) under the product name Actonel®.

b. Clinical Use of Bisphosphonates

Bisphosphonates are analogues of pyrophosphates and like them, are strongly bound to hydroxyapatite on the bone surface. Bisphosphonates are stable and reduce and inhibit activity of osteoclasts, cells functioning in the absorption and removal of osseous tissue.

Bisphosphonates may be divided into two types: nitrogenous and non-nitrogenous bisphosphonates. These two types of bisphosphonates inhibit activity of osteoclast cells by two different mechanisms. Non-nitrogenous bisphosphonates (such as clodronate, etidronate, or tiludronate) are metabolized in the cell to compounds that compete with adenosine triphosphate (ATP) in the cellular energy metabolism. This in turn causes the apoptosis of osteoclasts and leads to an overall decrease in the breakdown of bone. Nitrogenous bisphosphonates (such as pamidronate, neridronate, olpadronate, alendronate, ibandronate, zoledronate, risedronate, incadronate, or minodronate) act on bone metabolism by binding and blocking the enzyme farnesyl diphosphate synthase (FPPS) in the HMG-CoA reductase pathway (also known as the mevalonate pathway).

The clinical use of bisphosphonates is based on their ability to inhibit bone resorption. Thus, the main indications for their use include diseases with high bone remodeling, such as Paget's disease of bone, osteoporosis, metastatic bone diseases, and malignant and nonmalignant hypercalcemia.

The primary effect of bisphosphonates is the inhibition of bone resorption through cellular mechanisms that effect osteoclast attachment to bone, osteoclast precursor differentiation, and osteoclast survival. The anti-resorptive effect of bisphosphonates is also mediated through effects on the osteoblast.

The anticancer effects of bisphosphonates appear to involve modifying the bone surface, altering the bone microenvironment, inhibiting specific enzymatic pathways, and inducing apoptosis in osteoclast and tumor cells. The role of bisphosphonates as anti-cancer agents continues to expand and new and more potent bisphosphonates are continually being introduced into clinical practice. Exemplary clinical uses of bisphosphosphonates are discussed below.

For example, zoledronate has been used to prevent skeletal fractures in patients with cancers such as multiple myeloma and prostate cancer. It can also be used to treat hypercalcemia of malignancy and can be helpful for treating pain from bone metastases. Alendronate has been shown to increase bone mineral density (BMD), prevent radiographically defined (morphometric) vertebral fractures and positively affect morphometric as well as clinically evident fractures in postmenopausal women with low bone mass. Clodronate has been shown to inhibit increases in bone resorption and to prevent bone loss due to the menopause and during immobilization. Short-term and long-term studies indicate that clodronate stops bone loss at the lumbar spine in patients with vertebral osteoporosis. Clodronate treatment has also been shown to decrease bone turnover and to attenuate cancer-related bone morbidity. Etidronate has been shown to be suitable for treatment of patients with corticosteroid induced osteoporosis as it prevents loss of vertebral bone density in these patients. Pamidronate has been shown to be an effective agent for treatment of Paget's disease of bone, and also effective for treatment of osteoporosis. Tiludronate has been shown to be effective in reducing bone resorption in several metabolic bone diseases without inducing mineralization defects. The clinical development of tiludronate for the treatment of Paget's disease of bone has shown that tiludronate is equally suitable for treatment of osteoporosis. Ibandronate has been preferentially used for treatment of postmenopausal osteoporosis. Neridronate is a third generation amino-bisphosphonate shown to be useful for treatment and prevention of osteoporosis as well as collagen disease. Risedronate is a third generation bisphosphonate shown to be useful for treatment and prevention of osteoporosis. Olpadronate, incadronate, and minodronate are under development for similar therapeutic use as described for other bisphosphonates. It is generally understood in the art that, subject to differences in potency and dose-limiting toxicity, that bisphosphonates are generally interchangeable and are useful for treating a wide range of conditions associated with bone loss, including bone cancer and bone metastases.

### 3. Pharmaceutical Compositions of Bisphosphonates

When administered to an animal, such as a human, a bisphosphonate may be administered as a pharmaceutical composition. Any pharmaceutically acceptable form of a bisphosphonate may be used, for example, free acid or a pharmaceutically acceptable salt or ester thereof. Pharmaceutically acceptable salts, for instance, include sodium, potassium, calcium, magnesium, ammonium, sulfate, lactate, acetate, stearate, hydrochloride, tartrate, maleate, and the like. In certain embodiments, the bisphosphonate is selected from ibandronate, zoledronate, and risedronate, or a pharmaceutically acceptable salt or ester or a mixture thereof. In an exemplary embodiment, the bisphosphonate is zoledronate, a pharmaceutically acceptable salt or ester of zoledronate or a mixture thereof.

While most bisphosphonates for oral and intravenous administration are formulated as water-soluble salts (such as a disodium salt) to enhance their absorption and bioavailability, the drug delivery devices of invention can effectively deliver low-solubility salts of a bisphosphonate.

In certain embodiments, a bisphosphonate is complexed with a divalent or multivalent metal cation (such as Mg²⁺, Ca²⁺, Zn²⁺, or Mn²⁺) to form a low-solubility salt of the bisphosphonate. In general, “low solubility” means that the substance is only very slightly soluble in a medium (e.g., aqueous solutions having pH in the range of about 5 to about 8, and in particular physiologic solutions, such as blood, blood plasma, etc.). Some low-solubility bisphosphonates will have solubilities of less than about 1 mg/mL in the medium, less than about 100 μg/mL, preferably less than about 20 μg/mL, more preferably less than about 15 μg/mL, and even more preferably less than about 10 μg/mL. Solubility in water is measured at a temperature of 25° C. as measured by the procedures set forth in the 1995 USP, unless otherwise stated. According to the invention, bisphosphonates and/or salts which are slightly soluble (about 10 mg/mL to about 1 mg/mL), very slightly soluble (about 1 mg/mL to about 0.1 mg/mL) and practically insoluble or insoluble compounds (less than about 0.1 mg/mL, preferably less than about 0.01 mg/mL) are contemplated.

In certain embodiments, a bisphosphonate may have low solubility in the physiological fluid immediately surrounding the implanted/inserted drug delivery device. In such embodiments, the rate of release of the bisphosphonate from the drug delivery device, may be controlled by the solubility of the bisphosphonate in such surrounding fluid (i.e., the lower the solubility of the bisphosphonate in the immediately surrounding fluid the lower its rate of release from the drug delivery device). In certain embodiments, the solubility of the bisphosphonate in the surrounding physiological fluid is slightly soluble or less.

Additionally, the pharmaceutical composition of the invention may comprise a bisphosphonate (such as zoledronate) and a pharmaceutically acceptable carrier. Pharmaceutically acceptable carriers are well known in the art. In a preferred embodiment, when such pharmaceutical compositions are for human administration, the composition is pyrogen free, or substantially pyrogen free. The carrier(s) can be chosen, for example, to effect delayed release of an agent.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient.

### 4. Sustained Release Drug Delivery Devices

In certain aspects, the invention provides a novel drug delivery device for administration of a bisphosphonate to a patient, wherein the drug delivery device comprises a drug core comprising a bisphosphonate, preferably a low-solubility salt of the bisphosphonate. In certain embodiments, the drug delivery device provides sustained release of bisphosphonate at a rate sufficient to maintain a concentration of the bisphosphonate in the patient within a therapeutic range, and preferably at a substantially constant level.

In certain embodiments, the drug delivery device provides sustained release of a therapeutically effective dose of a bisphosphonate over a prolonged period, e.g., over a period of at least 1 day, at least 2 days, or even at least 1 week, at least 1 month, or at least 6 months, or at least 1 year. In an exemplary embodiment, the drug delivery device provides sustained release of a therapeutically effective dose of a bisphosphonate for six to twelve months.

In certain embodiments, the drug core of the device is disposed in a tube, wherein the tube is sized and formed of a material so that the tube is dimensionally stable.

There are several advantages of using a dimensionally stable tube that is capable of supporting its own weight. First, a dimensionally stable tube allows for significantly easier making and handling of the drug delivery device, because in contrast to previously described tubes with soft polymer coatings, a dimensionally stable tube can support its own weight in the presence or absence of the bisphosphonate drug core. Typically, a relatively small and short drug device is preferable for subcutaneous administration, because large devices may require more complex surgery for both implantation and removal. However, a longer device can be more efficient when used as a manufacturing intermediate, for example, by positioning the bisphosphonate drug core in a longer tube and cross-sectionally cutting the tube into sections to provide individual devices. Also, a drug slurry can readily be drawn into the tube when the tube is dimensionally stable, which in turn facilitates the fabrication of longer devices. Finally, because of the relative ease of manufacturing devices in accordance with the present invention, more than one drug core, optionally containing more than one drug, can be incorporated into a single device.

Second, a dimensionally stable tube makes it easier to obtain and maintain a desired release rate of the drug. In soft-structure devices, during use of the devices, the size, shape, or both, of the drug core change as drug diffuses out of the device, which in turn affects the release rate of the bisphosphonate, in particular if the diffusion rate depends on diffusion area of the drug core. Thus, when the tube which holds the drug core is sufficiently strong or rigid to maintain a relatively constant diffusion area, the diffusion rate from the device does not change substantially as the bisphosphonate drug core dissolves. By way of example and not of limitation, an exemplary method of ascertaining if the tube is sufficiently rigid is to form a device and measure the diffusion rate of the drug from the device over time. If the diffusion rate changes more than, for example, 20%, 30%, or 40%, or 50%, or 60%, or 70%, or 80%, of the diffusion rate expected based on the chemical potential gradient across the device at any particular time, the tube has changed shape and is not sufficiently rigid. Another exemplary test is to visually inspect the device as the drug diffuses over time, looking for signs that the tube has collapsed in part or in full.

In certain embodiments, the tube is permeable to passage of the bisphosphonate, while in others, the tube is impermeable to passage of the bisphosphonate. The tube has first and second ends, and preferably at least one of the first or second ends is permeable to passage of the bisphosphonate. As used herein, the term “permeable” is intended to mean permeable or substantially permeable to a substance (e.g., a bisphosphonate). As used herein, the term “impermeable” is intended to mean impermeable or substantially impermeable to substance (e.g., a bisphosphonate). In particular, an “impermeable” layer, as described in certain exemplary embodiments, will not allow passage of an effective therapeutic agent at a rate required to obtain the desired local or systemic physiological or pharmacological effect. Permeability is necessarily a relative term. It will be appreciated that in certain cases, a member (e.g., an end cap, or a membrane) may be permeable to a bisphosphonate, and also substantially control a rate at which the bisphosphonate diffuses or otherwise passes through the member. Consequently, a permeable member may also be a release-rate-limiting or release-rate-controlling member, and in certain circumstances, permeability of such a member may be one of the most significant characteristics controlling release rate for a device. Thus, if part of a tube is permeable and the rest of the device is impermeable, it is contemplated that, even though some drug may pass through the impermeable area of the tube, the drug will predominately be released through the permeable part of the tube.

In certain embodiments, the drug delivery device may comprise a permeable member (e.g., a permeable coating) that covers at least a portion of the tube first end and/or second end, wherein the permeable members allow diffusion of a bisphosphonate out of the drug core through the tube first and/or second ends. In other embodiments, the drug delivery device comprises permeable end caps abutting the tube first end and/or second end. In certain embodiments, the caps have a radial exterior surface and an end surface opposite the drug core. The caps may further comprise an outer layer that covers at least part of the radial exterior surface and at least a part of the cap's end surface.

In certain embodiments the drug delivery device further comprises an outer layer that completely surrounds all or a portion of the tube. In certain embodiments, the outer layer may be permeable, while in other embodiments, the outer layer may be impermeable.

The use of outer layers or other coverings for the ends of the tube provides flow resistance to reverse flow, i.e., flow back into the device. The outer layer or other covering assists in preventing large proteins from solubilizing the drug in the drug reservoir and/or in preventing oxidation and protein lysis, as well as preventing other biological agents from entering the reservoir and eroding or metabolizing the drug therein.

In selecting the permeability characteristics of the various components of the subject device, one of skill in the art will appreciate that it will typically be desirable to provide a path from the drug core to the exterior of the device that is either unobstructed or that is blocked by a permeable material. It is generally undesirable to surround the drug core with impermeable material. However, in certain embodiments, it may be advantageous to choose a biodegradable impermeable material for a portion of the device, such that upon degradation, the drug can elute from the drug core to the exterior of the device, thereby providing sustained release of the agent after an initial delay.

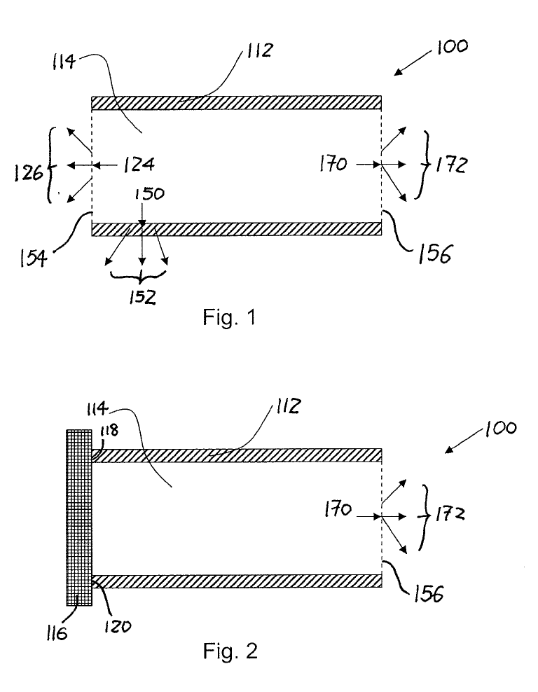

Turning now to the drawing figures, FIG. 1 illustrates a longitudinal cross sectional view of a drug delivery device 100 in accordance with the present invention. Device 100 includes a tube 112 and a drug core 114. The drug core may be formulated as a pure drug core, a drug in a polymer matrix, a drug in suspension, or any other suitable form.

In certain embodiments, tube 112 is permeable to the passage of a bisphosphonate disposed in the drug core 114; thus, the bisphosphonate diffuses out of the drug core along a first flow path 150 into portions of tube 112 that are immediately adjacent to the open area of the drug core. From the tube 112, the drug is free to diffuse along flow paths 152 and into the tissue or other anatomical structure in which device 100 is inserted or implanted.

In certain embodiments, tube 112 is impermeable to passage of a bisphosphonate disposed in the drug core 114, but at least one of the first end 154 or second end 156 of the tube is permeable to passage of the bisphosphonate. For example, a bisphosphonate may be free to diffuse along flow path 124 through permeable end 154, then along flow paths 126 into the tissue or other anatomical structure in which device 100 is inserted or implanted. Alternatively, a bisphosphonate may diffuse along flow path 170 through permeable end 156, then along flow paths 172.

FIG. 2 illustrates the device 100 with an optional cap 116 at one end of the tube, opposite end 156. Device 100 now includes a tube 112, a drug core 114, and a cap 116. Drug core 114 is positioned in the interior of tube 112. Cap 116 is positioned at one end of tube 112, and is joined to the tube at end 118, 120 of the tube. While cap 116 may extend radially beyond tube 112, as illustrated in FIG. 2, the cap may alternatively have substantially the same radial extent as, or a slightly smaller radial extent than, the tube, within the spirit and scope of the present invention.

When provided, cap 116 and tube 112 can be formed separately and assembled together, or the tube and the cap can be formed as a single, integral, monolithic element.

Tube 112 and cap 116 may be each independently permeable or impermeable to the passage of a bisphosphonate disposed in drug core 114, so that the bisphosphonate is free to diffuse through cap 116, tube 112, or tube end 156 in a similar fashion as described in FIG. 1. In an exemplary embodiment, tube 112 and cap 116 are impermeable but end 156 is permeable to the bisphosphonate contained in drug core 114. The bisphosphonate is free to diffuse through end 156, from the drug core along flow paths 170, 172, into the tissue or other anatomical structure in which device 100 is inserted or implanted.

Optionally, tube 112 may be closed off or sealed by a disk on the end of tube 112, opposite cap 116 (embodiments not shown in FIG. 2). The disk can be permeable or impermeable to the passage of a bisphosphonate disposed in drug core.

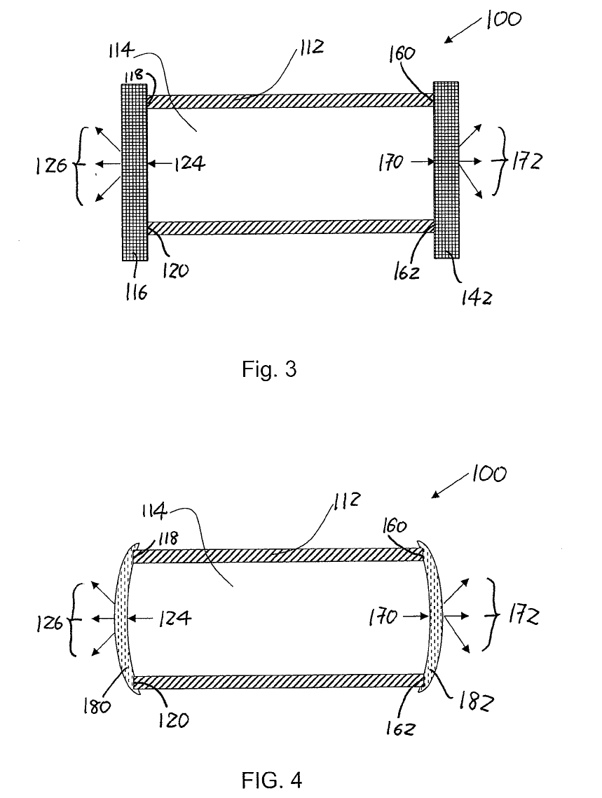

FIG. 3 illustrates a device 100 with two optional caps 116 and 142. As shown device 100 includes a tube 112, a drug core 114, and two end caps 116 and 142. Drug core 114 is positioned in the interior of tube 112. Cap 116 is positioned at the first end of tube 112, and is joined to the tube at end 118, 120 of the tube. Cap 142 is positioned at second end of tube 112, and is joined to the tube at end 160 and 162 of the tube. While caps 116 and 142 may extend radially beyond tube 112, as illustrated in FIG. 3, the cap may alternatively have substantially the same radial extent as, or a slightly smaller radial extent than, the tube, within the spirit and scope of the present invention. When provided, caps 116, 142 and tube 112 can be formed separately and assembled together, or the tube and the caps can be formed as a single, integral, monolithic element.

Tube 112, caps 116 and 142 may be each independently permeable or impermeable to the passage of a bisphosphonate disposed in drug core 114, so that the bisphosphonate is free to diffuse through the tube, or the cap, in a similar fashion as described in FIG. 1. For example, tube 112 may be impermeable, and an impermeable cap 142 may be positioned at the end of reservoir opposite a permeable cap 116, so that the bisphosphonate may diffuse through cap 116 from the reservoir along flow paths 124, 126, into the tissue or other anatomical structure in which device 100 is inserted or implanted. Cap 116 therefore preferably has a radial extent which is at least as large as the radial extent of drug core 114, so that the only diffusion pathway 124 out of the reservoir is through the cap. Alternatively, both caps 116 and 142 may be permeable, so that the bisphosphonate may diffuse through the caps 116 and 142 from the drug core along flow paths 124, 126, 170, and 172, into the tissue or other anatomical structure in which device 100 is inserted or implanted.

FIG. 4 illustrates a device 100 in which both ends of tube 112 are optionally coated with layers 180 and 182. As shown device 100 includes a tube 112, a drug core 114, and layers 180 and 182. Drug core 114 is positioned in the interior of tube 112. Layer 180 is positioned at the first end of tube 112, and is joined to the tube at end 118 and 120 of the tube. Layer 182 is positioned at second end of tube 112, and is joined to the tube at end 160 and 162 of the tube. While the layers may extend radially beyond tube 112, as illustrated in FIG. 4, the layers may alternatively have substantially the same radial extent as, or a slightly smaller radial extent than, the tube, within the spirit and scope of the present invention.

Tube 112 and layers 180 and 182 may be each independently permeable or impermeable to the passage of a bisphosphonate disposed in drug core 114, so that the bisphosphonate is free to diffuse through the tube, or the outer layer, in a similar fashion as described in FIG. 1. For example, tube 112 may be impermeable, and both layers 180 and 182 may be permeable, so that the bisphosphonate may diffuse through the layer 180 and 182 from the drug core along flow paths 124, 126, 170, and 172, into the tissue or other anatomical structure in which device 100 is inserted or implanted.

Alternatively, only one end of tube 112 may be coated with a layer (e.g., tube 112 coated with layer 180 at the first end only). The other end of the tube may be an open end, or closed off or sealed by a cap or a disk (not shown in FIG. 4). As discussed before, the tube, the layer, the cap (if any), and the disk (if any) may be each independently permeable or impermeable to the passage of a bisphosphonate disposed in drug core.

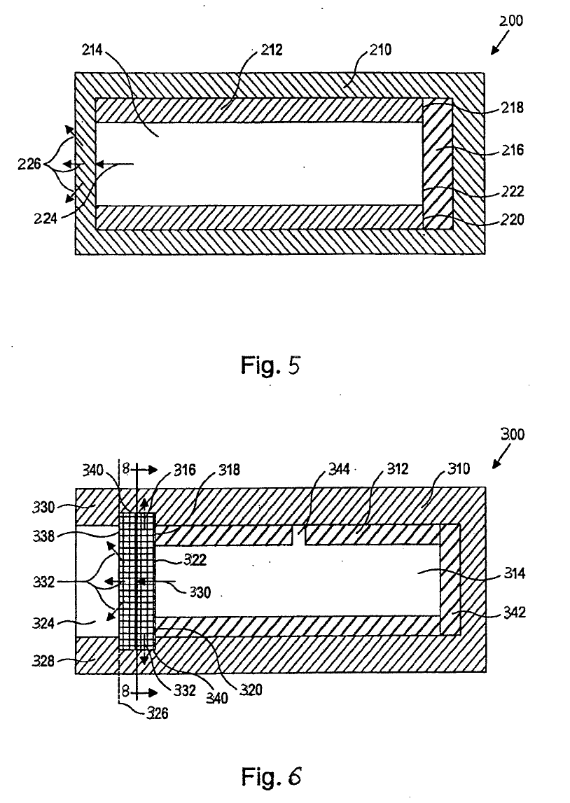

In certain embodiments, the drug delivery devices of the present invention further comprise an outer layer, an inner tube, and a bisphosphonate disposed in a drug core. Exemplary embodiments of devices of the invention with outer layers and inner tubes are shown in FIGS. 5-8.

FIG. 5 illustrates a longitudinal cross sectional view of a drug delivery device 200 in accordance with the present invention. Device 200 includes an outer layer 210, an inner tube 212, a drug core 214, and an inner cap 216. Outer layer 210 is preferably a permeable layer, that is, the outer layer is permeable to the drug contained within drug core 214. Cap 216 is positioned at one end of tube 212. Cap 216 is preferably formed of an impermeable material, that is, the cap is not permeable to the drug contained within drug core 214. Cap 216 is joined at end 218, 220 of inner tube 212, so that the cap and the inner tube together close off a space in the tube in which drug core 214 is positioned, and together the cap and inner tube form a cup- or vessel-shaped member Inner tube 212 and cap 216 can be formed separately and assembled together, or the inner tube and the cap can be formed as a single, integral, monolithic element.

Outer layer 210 at least partially, and preferably completely, surrounds both tube 212 and cap 216, as illustrated in FIG. 5. While it is sufficient for outer layer 210 to only partially cover tube 212 and cap 216, and in particular the opposite ends of device 200, the outer layer is preferably formed to completely envelop both the tube and cap to provide structural integrity to the device, and to facilitate further manufacturing and handling because the device is less prone to break and fall apart. While FIG. 5 illustrates cap 216 having an outer diameter the same as the outer diameter of inner tube 212, the cap can be sized somewhat smaller or larger than the outer diameter of the inner tube within the spirit and scope of the present invention.

Drug core 214 is positioned inside inner tube 212. A first end 222 abuts against cap 216, and is effectively sealed by the cap from diffusing drug therethrough. On the end of drug core 214 opposite cap 216, the drug core is preferably in direct contact with outer layer 210. As will be readily appreciated by one of ordinary skill in the art, as drug is released from drug core 214, the drug core may shrink or otherwise change shape, and therefore may not fully or directly contact outer layer 210 at the end of the drug core opposite cap 216. As outer layer 210 is permeable to the drug in drug core 214, the drug is free to diffuse out of the drug core along a first flow path 224 into portions of outer layer 210 immediately adjacent to the open end of the drug core. From outer layer 210, the drug is free to diffuse along flow paths 226 out of the outer layer and into the tissue or other anatomical structure in which device 200 is inserted or implanted. Optionally, holes can be formed through inner layer 212 to add additional flow paths 226 between drug core 214 and permeable outer layer 210.

FIG. 5 illustrates only the positions of the several components of device 200 relative to one another, and for ease of illustration shows outer layer 210 and inner tube 212 as having approximately the same wall thickness. While the walls of outer layer 210 and inner tube 212 may be of approximately the same thickness, the inner tube's wall thickness can be significantly thinner or thicker than that of the outer layer within the spirit and scope of the present invention. Additionally, device 200 is preferably cylindrical in shape, for which a transverse cross-section (not illustrated) will show circular cross-sections of the device. While it is preferred to manufacture device 200 as a cylinder with circular cross-sections, it is also within the scope of the present invention to provide cap 216, drug core 214, inner tube 212, and/or outer layer 210 with other cross-sections, such as ovals, ellipses, rectangles, including squares, triangles, as well as any other regular polygon or irregular shapes. Furthermore, device 200 can optionally further include a second cap (not illustrated) on the end opposite cap 216; such a second cap could be used to facilitate handling of the device during fabrication, and would include at least one through hole for allowing drug from drug core 214 to flow from the device.

FIG. 6 illustrates a device 300 in accordance with another exemplary embodiment of the present invention. Device 300 includes an impermeable inner tube 312, a drug core 314, and a permeable cap 316. Device 300 optionally and preferably includes an impermeable outer layer 310, which adds mechanical integrity and dimensional stability to the device, and aids in manufacturing and handling the device. Cap 316 is positioned at one end of inner tube 312, and is joined to the inner tube at end 318, 320 of the inner tube. While cap 316 may extend radially beyond inner tube 312, as illustrated in FIG. 6, the cap may alternatively have substantially the same radial extent as, or a slightly smaller radial extent than, the inner tube, within the spirit and scope of the present invention. As cap 316 is permeable to the bisphosphonate contained in drug core 314, the bisphosphonate is free to diffuse through the cap from the drug core. Cap 316 therefore preferably has a radial extent which is at least as large as the radial extent of drug core 314, so that the only diffusion pathway 330 out of the drug core is through the cap. On the end of inner tube 312 opposite cap 316, the inner tube is closed off or sealed only by outer layer 310, as described below. Optionally, an impermeable cap 342, which can take the form of a disc, is positioned at the end of drug core opposite cap 316. When provided, cap 342 and inner tube 312 can be formed separately and assembled together, or the inner tube and the cap can be formed as a single, integral, monolithic element.

Outer tube or layer 310, when provided, at least partially, and preferably completely surrounds or envelopes inner tube 312, drug core 314, cap 316, and optional cap 342, except for an area immediately adjacent to the cap which defines a port 324. Port 324 is, in preferred embodiments, a hole or blind bore which leads to cap 316 from the exterior of the device. As outer layer 310 is formed of a material which is impermeable to the agent in drug core 314, the ends of inner tube 312 and drug core 314 opposite cap 316 are effectively sealed off, and do not include a diffusion pathway for the agent to flow from the drug core. According to a preferred embodiment, port 324 is formed immediately adjacent to cap 316, on an end 338 of the cap opposite end 322 of drug core 314. Cap 316 and port 324 therefore include diffusion pathways 330, 332, through the cap and out of device 300, respectively.

While port 324 in the embodiment illustrated in FIG. 6 has a radial extent which is approximately the same as inner tube 312, the port can be sized to be larger or smaller, as will be readily apparent to one of ordinary skill in the art. For example, instead of forming port 324 radially between portions 328, 330 of outer layer 310, these portions 328, 330 can be removed up to line 326, to increase the area of port 324. Port 324 can be further enlarged, as by forming outer layer 310 to extend to cover, and therefore seal, only a portion or none of the radial exterior surface 340 of cap 316, thereby increasing the total surface area of port 324 to include a portion or all of the outer surface area of the cap 316.

In accordance with yet another embodiment of the present invention, port 324 of device 300 can be formed immediately adjacent to radial external surface 340 of cap 316, in addition to or instead of being formed immediately adjacent to end 338 of the cap 316. As illustrated in FIG. 8, port 324 can include portions 334, 336, which extend radially away from cap 316. These portions can include large, continuous, circumferential and/or longitudinal portions 336 of cap 316 which are not enveloped by outer layer 310, illustrated in the bottom half of FIG. 8, and/or can include numerous smaller, circumferentially spaced apart portions 334, which are illustrated in the top half of FIG. 8. Advantageously, providing port 324 immediately adjacent to radial external surface 340 of cap 316, as numerous, smaller openings 334 to cap 316, allows numerous alternative pathways for the agent to diffuse out of device 300 in the event of a blockage of portions of the port. Larger openings 336, however, benefit from a relative ease in manufacturing, because only a single area of cap 316 need be exposed to form port 324.

According to yet another embodiment of the present invention, cap 316 is formed of an impermeable material and outer layer 310 is formed of a permeable material. A hole or holes are formed, e.g., by drilling, through one or more of inner layer 312, cap 342, and cap 316, which permit drug to be released from drug core 314 through outer layer 310. According to another embodiment, cap 316 is eliminated as a separate member, and permeable outer layer 310 completely envelopes inner tube 312 and cap 342 (if provided). Thus, the diffusion pathways 330, 332 are through outer layer 310, and no separate port, such as port 324, is necessary. By completely enveloping the other structures with outer layer or tube 310, the system 300 is provided with further dimensional stability. Further optionally, cap 316 can be retained, and outer layer 310 can envelop cap 316 as well.

According to yet another embodiment of the present invention, inner tube 312 is formed of a permeable material, outer layer 310 is formed of an impermeable material, and cap 342 is formed of either a permeable or an impermeable material. Optionally, cap 342 can be eliminated. As described above, as outer layer 310 is impermeable to the agent in drug core 314, cap 316, port 324, and optional ports 334, 336 allow passage of the agent out of device 300.

The shape of device 300 can be, in a manner similar to that described above with respect to device 200, any of a large number of shapes and geometries. Furthermore, both device 200 and device 300 can include more than one drug core 214, 314, included in more than one inner tube 212, 312, respectively, which multiple drug cores can include diverse or the same agent or drug for diffusion out of the device. In device 300, multiple drug cores 314 can be positioned to abut against only a single cap 316, or each drug core 314 can have a dedicated cap for that drug core. Such multiple drug cores can be enveloped in a single outer layer 210, 310, as will be readily appreciated by one of ordinary skill in the art.

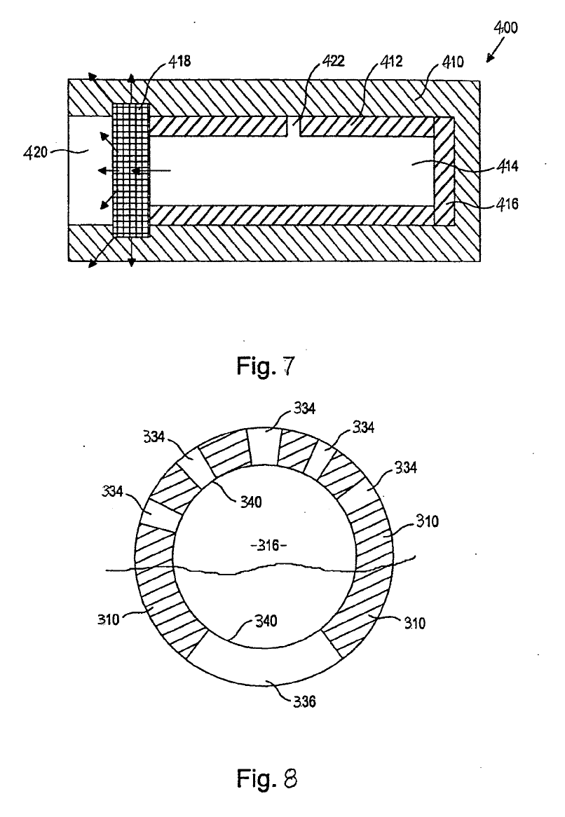

Turning now to FIG. 7, FIG. 7 illustrates a device 400 in accordance with an exemplary embodiment of the present invention. Device 400 includes a permeable outer layer 410, an impermeable inner tube 412, a drug core 414, an impermeable cap 416, and a permeable cap 418. A port 420 communicates cap 418 with the exterior of the device, as described above with respect to port 324 and cap 316. Inner tube 412 and cap 416 can be formed separately and assembled together, or the inner tube and the cap can be formed as a single, integral, monolithic element. The provision of permeable outer layer 410 allows the therapeutic agent in reservoir or drug core 414 to flow through the outer layer in addition to port 420, and thus assists in raising the overall delivery rate. Of course, as will be readily appreciated by one of ordinary skill in the art, the permeability of cap 418 is the primary regulator of the drug delivery rate, and is accordingly selected. Additionally, the material out of which outer layer 410 is formed can be specifically chosen for its ability to adhere to the underlying structures, cap 416, tube 412, and cap 418, and to hold the entire structure together. Optionally, a hole or holes 422 can be provided through inner tube 412 to increase the flow rate of drug from drug core 414.

In certain embodiments, the drug delivery devices of the invention comprise an inner tube that is sized and formed of a material so that the inner tube is dimensionally stable. The advantages of using a dimensionally stable tube has been discussed before.

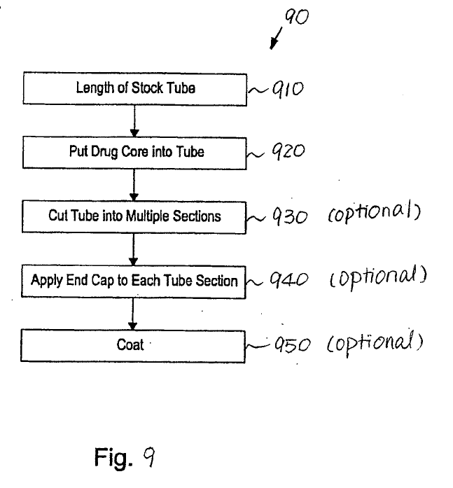

By way of example only and not of limitation, FIG. 9 illustrates a process of forming devices of the invention according to an exemplary embodiment of the invention. As shown in FIG. 9, in process 90, a length of tube stock material 910 may be taken as the starting material. In the subsequent step 920, a drug core may be inserted, injected, or otherwise positioned into the open end of tube, depending on how viscous the drug core material is when positioned in the tube. If the drug core is relatively stiff, i.e., is very viscous or solid, the drug core can be inserted into the tube, as with a plunger, pushrod, or the like. If the drug core is relatively flaccid or fluid, i.e., is not very viscous, the drug core can be poured, injected, or drawn into the tube (e.g., by vacuum). Then, in optional step 930, the length of tube, including the drug core, may be cut into multiple sections, to form one or more tubes of preferred length. Afterwards, in an optional step 940, the tube may be sealed to form a closed, cup- or vessel-like structure, for example, by joining a cap at one end of the tube or coating one or both open ends with a polymer membrane (e.g., permeable or impermeable). Because of the relative rigidity of the tube, the tube (and the cap, if any) can be handled with relative ease, because the tube is sized and formed of a material so that it is capable of supporting its own weight without collapsing. Thereafter, in another optional step 950, the tube can be coated with a layer (such as end layers illustrated in FIG. 4, or outer layers illustrated in FIGS. 5-7).

According to yet another embodiment of a process for manufacturing in accordance with the present invention, a drug core can be inserted into a mold, along with the cap (if any), and the tube can be molded around the drug core and the cap (if any). Further alternatively, a cap can be formed integrally with the tube.

A large number of materials can be used to construct the devices of the present invention. The only requirements are that they are inert, non-immunogenic, and of the desired permeability, as described above.

Materials that may be suitable for fabricating devices of the invention (such as 100, 200, 300, and 400 described above) include naturally occurring or synthetic materials that are biologically compatible with body fluids or tissues, and do not rapidly dissolve in body fluids with which the material will come in contact. The use of rapidly dissolving materials is preferably avoided since dissolution of the outer surface of the device (e.g., tube 112, or outer layers 210, 310, or 410) would affect the constancy of the drug release, as well as the capability of the system to remain in place for a prolonged period of time. To the extent biodegradable materials are selected for one or more structural components of the device, particularly for a rigid tube, such materials are generally preferably configured to remain substantially intact throughout the period of drug release from the device. To the extent biodegradable materials are selected for one or more structural components of the device, particularly for a rigid tube, such materials are generally preferably configured to remain substantially intact throughout the period of drug release from the device.

Naturally occurring or synthetic materials that are biologically compatible with body fluids and tissues and do not rapidly dissolve in body fluids which the material will come in contact include, but are not limited to: ethyl vinyl acetate, polyvinyl acetate, cross-linked polyvinyl alcohol, cross-linked polyvinyl butyrate, ethylene ethylacrylate copolymer, polyethyl hexylacrylate, polyvinyl chloride, polyvinyl acetals, plasticized ethylene vinylacetate copolymer, polyvinyl alcohol, polyvinyl acetate, ethylene vinylchloride copolymer, polyvinyl esters, polyvinylbutyrate, polyvinylformal, polyamides, polymethylmethacrylate, polybutyl-methacrylate, plasticized polyvinyl chloride, plasticized nylon, plasticized soft nylon, plasticized polyethylene terephthalate, natural rubber, polyisoprene, polyisobutylene, polybutadiene, polyethylene, polytetrafluoroethylene, polyvinylidene chloride, polyacrylonitrile, cross-linked polyvinylpyrrolidone, polytrifluorochloroethylene, chlorinated polyethylene, poly(1,4′-isopropylidene diphenylene carbonate), vinylidene chloride, acrylonitrile copolymer, vinyl chloride-diethyl fumarate copolymer, silicone rubbers (especially medical grade polydimethylsiloxanes), ethylene-propylene rubber, silicone-carbonate copolymers, vinylidene chloride-vinyl chloride copolymer, vinyl chloride-acrylonitrile copolymer, vinylidene chloride-acrylonitrile copolymer, gold, platinum, (surgical) stainless steel, and silicon.

For example, the tube may be made of a polymer, a metal, or silicon. The permeable outer layer (e.g., 210) may be made of a permeable polymer such as polyethylene glycol (PEG), poly(dl-lactide-co-glycolide) (PLGA), poly(vinyl alcohol) (PVA), poly(glycolic acid) (PGA), or a copolymer thereof.

Additionally, materials for the tube and the drug core of the drug delivery device may be selected to be stable so that, for the desired life of the delivery device (e.g., during the release period for the drug delivery device), the materials do not significantly erode, and the pore size of the materials does not change.

Alternatively, the materials may be selected so that, after the drug delivery device has released the drug for a predetermined amount of time, the drug delivery device erodes in situ, i.e., is bioerodible, at rates that control, or contribute to control of, the release rate of a bisphosphonate. It will be appreciated that other materials, such as additional coatings on some or all of the device may be similarly selected for their bioerodible properties. Exemplary permeable bioerodible materials include polyethylene glycol (PEG), poly(dl-lactide-co-glycolide) (PLGA), poly(vinyl alcohol) (PVA), or poly(glycolic acid) (PGA), or a copolymer thereof. Exemplary impermeable bioerodible materials include ethylene vinyl acetate polymer (EVA), polyalkyl cyanoacrylate, polyurethane, nylon, or a copolymer thereof.

More specifically, tubes 112 of devices 100, and outer layer 210, 310, and 410 of devices 200, 300 and 400 may be made of any of the above-listed polymers or any other polymer which is biologically compatible with body fluids or tissues, does not rapidly dissolve in body fluids with which the material will come in contact, and is essentially impermeable to the passage of the effective agent.

When tube 112, or inner tubes 212, 312, and 412 are selected to be impermeable to the passage of a bisphosphonate from the drug core or reservoir to adjacent portions of the device, as described above, the purpose is to block the passage of the bisphosphonate to those portions of the device, and thus control the release of the agent out of the drug delivery device permeable caps or permeable outer layers of the device.

The composition of a permeable tube, a permeable cap, or a permeable outer layer of the devices of the invention (e.g., a polymer) is preferably selected so as to allow the above-described controlled release. For example, outer layer 210 and caps 316, 418 of FIGS. 5-8 are preferably permeable to the passage of the bisphosphonate at a rate that the bisphosphonate is released at a therapeutically effective dosage. The preferred composition will vary depending on such factors as the active ingredient, the desired rate of control, and the mode of administration. The identity of the active ingredient is important since the size of the molecule, for instance, is critical in determining the rate of release of the bisphosphonate.

Caps 216, 342, 416 are preferably essentially impermeable to the passage of the effective agent and may cover a portion of the inner tube not covered by the outer layer. The physical properties of the material, preferably a polymer, used for the caps can be selected based on their ability to withstand subsequent processing steps (such as heat curing) without suffering deformation of the device. For example, the material, e.g., polymer, for impermeable outer layer 310 can be selected based on the ease of coating inner tube 312.

Tubes, outer layers, and/or caps of the device of the present invention are preferably biologically compatible with body fluids and tissues, essentially insoluble in body fluids which the material will come in contact.

A bisphosphonate diffuses in the direction of lower chemical potential, i.e., toward the exterior surface of the device. At the exterior surface of the device, equilibrium is again established. For example, when the conditions on both sides of outer layer 210 or permeable caps 316, 418 are maintained constant, a steady state flux of a bisphosphonate will be established in accordance with Fick's Law of Diffusion. The rate of passage of the bisphosphonate through the material by diffusion is generally dependent on the solubility of the bisphosphonate therein, as well as on the thickness of the wall. This means that selection of appropriate materials for fabricating outer layer 210 and permeable cap 316 will be dependent on the particular bisphosphonate to be used and the desired rate of release.

The rate of diffusion of the effective agent through a polymeric layer of the present invention may be determined via diffusion cell studies carried out under sink conditions. In diffusion cell studies carried out under sink conditions, the concentration of drug in the receptor compartment is essentially zero when compared to the high concentration in the donor compartment. Under these conditions, the rate of drug release is given by:

Q/t=(D·K·A·DC)/h

where Q is the amount of drug released, t is time, D is the diffusion coefficient, K is the partition coefficient, A is the surface area, DC is the difference in concentration of the drug across the membrane, and h is the thickness of the membrane.

In the case where the bisphosphonate diffuses through the layer via water-filled pores, there is no partitioning phenomenon. Thus, K can be eliminated from the equation. Under sink conditions, if release from the donor side is very slow, the value DC is essentially constant and equal to the concentration of the donor compartment. Release rate therefore becomes dependent on the surface area (A), thickness (h), and diffusivity (D) of the membrane. In the construction of the devices of the present invention, the size (and therefore, surface area) is mainly dependent on the size of the bisphosphonate.

Thus, permeability values may be obtained from the slopes of a Q versus time plot. The permeability P can be related to the diffusion coefficient D by:

P=(K·D)/h

Once the permeability is established for the material permeable to the passage of the bisphosphonate, the release rate may be further slowed by coating some portion of the surface area with a material impermeable (or at least less permeable) to the passage of the bisphosphonate. Progressively coating additional portions of the available surface area of the permeable material increasingly reduces the release rate until the desired release rate is obtained.

Exemplary microporous materials suitable for use as tubes, caps, and outer layers of the devices of the invention are described, for instance, in U.S. Pat. No. 4,014,335, which is incorporated herein by reference in its entirety. These materials include cross-linked polyvinyl alcohol, polyolefins or polyvinyl chlorides or cross-linked gelatins; regenerated, insoluble, non-erodible cellulose, acylated cellulose, esterified celluloses, cellulose acetate propionate, cellulose acetate butyrate, cellulose acetate phthalate, cellulose acetate diethyl-aminoacetate; polyurethanes, polycarbonates, and microporous polymers formed by co-precipitation of a polycation and a polyanion modified insoluble collagen. Cross-linked polyvinyl alcohol is preferred for both outer layer 210 and permeable caps 316, 418.

The devices of the present invention may be made in a wide variety of ways, portions of which are described in greater detail above. For example, for devices 200, 300, and 400, once the drug core and tube have been assembled along with caps 216, 342, 316, or 418, the outer layer may be applied. The outer layer may be applied by dipping all or part of the device one or more times in a solution containing the desired polymer. Optionally, the outer layer may be applied by dropping, spraying, brushing or other means of coating the outer surface of the device with the polymer solution. When using a polyvinyl alcohol solution to obtain the outer layer, the desired thickness may be obtained by applying several coats. Each coat may be dried prior to applying the next coat. Finally, the device may be heated to adjust the permeability of outer layer 210 or caps 316 or 418.

Impermeable polymer layers in devices in accordance with the present invention are preferably thick enough to prevent release of drug across them except for the area not covered, e.g., port 324. Due to the desirability of minimizing the size of the implantable devices, the thickness of an impermeable layer therefore can be between about 0.01 and about 2 millimeters, preferably between about 0.01 and about 0.5 millimeters, most preferably between about 0.01 and about 0.2 millimeters.

Caps 216, 342, when formed of an impermeable material, are preferably also thick enough to prevent drug release across it. Due to the desirability of minimizing the size of the implants, the thickness of the impermeable cap 216 can be between about 0.01 and about 2 millimeters, preferably between about 0.01 and about 0.5 millimeter, most preferably between about 0.01 and about 0.2 millimeter.

The above description of how to make the devices of the present invention is merely illustrative and should not be considered as limiting the scope of the invention in any way, as various compositions are well known by those skilled in the art. In particular, the methods of making the device depends on the identity of the active agent and polymers selected. Given the active agent, the composition of the outer layers, the inner tube, the plug, and the cap, one skilled in the art could easily make the devices of the present invention using conventional coating techniques. Additional exemplary methods and processes useful for making drug delivery devices of invention may be found, for example, in PCT publication Nos. WO 03/094888 and WO 2005/051234 (incorporated by reference herein in each of their entireties).

### 4. Treatment or Prevention of Bone Loss

The invention provides methods of treating or preventing bone loss in a patient comprising implanting the drug delivery device of the invention, in particular, a device comprising a drug core disposed in a tube, wherein the drug core comprises a bisphosphonate. For example, the drug delivery device may be implanted to an individual subcutaneously to obtain the desired local or systemic effect of the bisphosphonate at the desired concentration.

The devices and methods of the invention may be utilized for treatment or prevention of bone loss in an individual. In certain embodiments, the individual is suffering from osteoporosis, Paget's disease, or bone metastases. In certain embodiments, the individual is a mammal such as a human, or a non-human mammal.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

Preferably, a bisphosphonate is formulated in a suitable pharmaceutical composition so that after subcutaneous implantation, the device provides sustained release of a therapeutically effective dose of a bisphosphonate for six to twelve months. The drug delivery device may be administered for a sufficient period of time and under conditions to allow treatment of the disease state of concern. In an exemplary embodiment, the drug delivery device comprises from about 10 mg to about 20 mg of a bisphosphonate.

For localized drug delivery, the devices may be surgically implanted or injected at or near the site of action. This is the case for devices of the present invention used in treating ocular conditions, primary tumors, rheumatic and arthritic conditions, and chronic pain.

For systemic relief, the devices may be implanted subcutaneously, intramuscularly, intraarterially, intrathecally, or intraperitoneally. This is the case when devices are to give sustained systemic levels and avoid premature metabolism.

The patient receiving this treatment is any animal in need, including primates, in particular humans, and other mammals such as equines, cattle, swine and sheep; and poultry and pets in general.

While the above described embodiments of the invention are described in terms of preferred ranges of the amount of effective agent, and preferred thicknesses of the preferred layers, these preferences are by no means meant to limit the invention. As would be readily understood by one skilled in the art, the preferred amounts, materials and dimensions depend on the method of administration, the effective agent used, the polymers used, the desired release rate and the like. Likewise, actual release rates and release duration depend on a variety of factors in addition to the above, such as the disease state being treated, the age and condition of the patient, the route of administration, as well as other factors which would be readily apparent to those skilled in the art. All of the foregoing U.S. patents and other publications are expressly incorporated by reference herein in each of their entireties.

From the foregoing description, one of ordinary skill in the art can easily ascertain the essential characteristics of the instant invention, and without departing from the spirit and scope thereof, can make various changes and/or modifications of the invention to adapt it to various usages and conditions. As such, these changes and/or modifications are properly, equitably and intended to be, within the full range of equivalence of the following claims.

### EXEMPLIFICATION

The invention now being generally described, it will be more readily understood by reference to the following examples, which are included merely for purposes of illustration of certain aspects and embodiments of the present invention, and are not intended to limit the invention.

Those skilled in the art will recognize, or be able to ascertain using no more than routine experimentation, many equivalents to the specific embodiments of the invention described herein. Such equivalents are intended to be encompassed by the following embodiments.

### Example 1

### 0.9 mm Bisphosphonate Implants

100 mg of magnesium zoledronate (9TC-26) and 70 μL of 10% PVA solution was mixed thoroughly and granulated. The granulation was air-dried overnight. The dried granules were ground to fine particles and blended with 200 μg of magnesium stearate. The blend was then compressed to rod shaped pellets (˜3 mg, 0.9 mm in diameter).

The pellets were inserted into precut 18G polyimide tubes, and the formed implants were divided into two groups: (A) 3 implants were both ends coated with 10% PVA solution, air dried. Coating was repeated one more time; (B) 6 implants were first sealed with silicone adhesive in one end and the other end was coated twice with 10% PVA solution.

After drying, all implants were placed in an oven and heated at 135° C. for 5 hours.

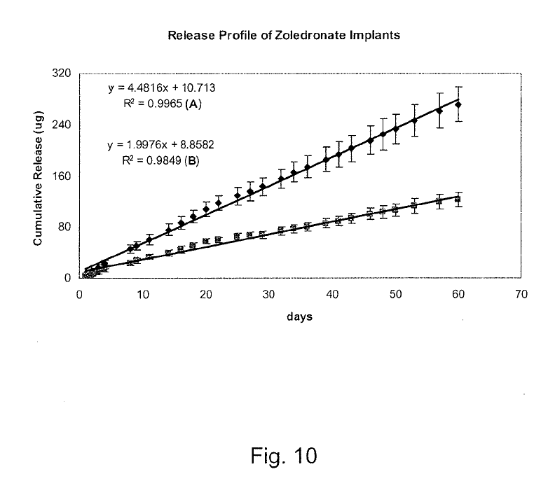

The release rate was tested by placing each implant in 1.0 mL of 0.1 M phosphate buffer (pH 7.4) in a 37° C. water bath. Samples were taken daily by replacing entire medium with fresh buffer. Drug amount released was determined by HPLC. FIG. 10 shows the release profile (measured as μg/day) of the 0.9 mm implants.

### Example 2

### 2.0 mm Bisphosphonate Implants

150 mg of magnesium zoledronate (9TC-26) and 150 μL of 10% PVA solution was mixed thoroughly and granulated. The granulation was air-dried overnight. The dried granules were ground to fine particles and blended with 300 μg of magnesium stearate. The blend was then compressed to rod shaped pellets (˜20 mg, 2.0 mm in diameter).

The pellets were dip coated with 10% PVA solution, air dried overnight, and then inserted into precut silicone tubes. The implants were placed in oven and heated at 135° C. for 5 hours.

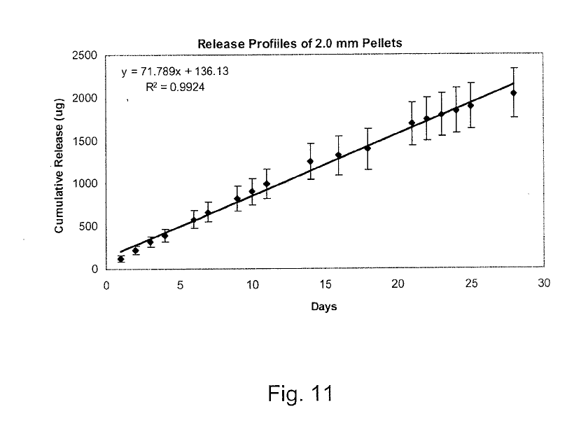

The release rate was tested by placing each implant in 5.0 mL of 0.1 M phosphate buffer (pH 7.4) in a 37° C. water-bath. Samples were taken daily by replacing entire medium with fresh buffer. Drug amount released was determined by HPLC. FIG. 11 shows the release profile (measured as μg/day) of the 2.0 mm implants.

### Example 3

### Sustained Release of an Exemplary Low Solubility Agent

An exemplary lipophilic/low-solubility agent, spironolactone, was used to examine the sustained release of a lipophilic/low-solubility agent when introduced into a physiological environment.

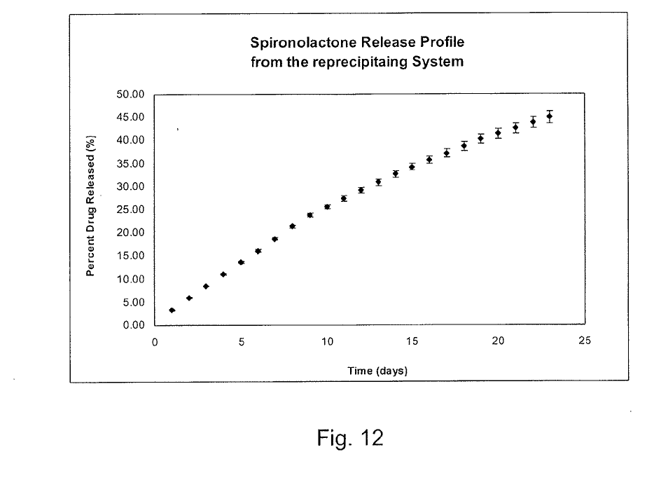

To prepare a stock solution, 150 mg spironolactone was dissolved in 500 μL of N-methyl-pyrrolidinone, a biocompatible organic solvent. 20 μL of spironolactone stock solution was then dispersed in 6 mL of release medium (50 mM phosphate buffer, pH 7.4). As expected, solid precipitate was formed in the release medium. FIG. 12 shows the release profile of spironolactone.

### Example 4

### Calcium Zoledronate

Zoledronic acid monohydrate (1.16 g) was suspended in 8 mL of water and treated with 4 mL of 1N sodium hydroxide to form a homogenous solution. A solution of calcium hydroxide (0.29 g) in 2 mL of water was then added. The mixture was cooled and the solid white product was filtered off, washed with water, and dried under high vacuum to yield 1.07 g (92%) of the product.

### Example 5

### Zinc Zoledronate

Zoledronic acid monohydrate (0.58 g) was suspended in 4 mL of water and 2 mL of 1N sodium hydroxide were added. The clear solution of the sodium salt was then treated with zinc chloride (0.136 g) dissolved in 2 mL of water to yield a heavy, white precipitate. The solid was filtered off, washed with water, and dried under high vacuum to give 0.56 g (92%) of the product.

### Example 6

### Calcium Ibandronate

Ibandronic acid (0.64 g) was dissolved in 5 mL of warm water. Solid calcium hydroxide (0.074 g) was added and the mixture was stirred at 60° C. for 30 min to give a clear solution. The solution was freeze dried to afford 0.7 g of the calcium salt.

### Example 7

### Magnesium Ibandronate

The salt was prepared as described above for calcium ibandronate using 0.64 g of ibandronic acid and 0.058 g of magnesium hydroxide. Yield 0.68 g

### Example 8

### Zinc Ibandronate

Ibandronic acid (0.64 g) was dissolved in 4 mL of water and 2 mL of 1N sodium hydroxide. A solution of zinc chloride (0.136 g) in 2 mL of water was added to give a white precipitate of the product. The solid was filtered off, washed with water, and dried to afford 0.55 g of the zinc salt.

### Example 9

### Benzathine Salt of Ibandronic Acid

A solution of ibandronic acid (0.32 g) in 14 mL of methanol was mixed with solution of benzathine (0.12 g) in 5 mL of methanol. The mixture was concentrated under vacuum and triturated with ethyl acetate to afford a white solid. The product was filtered off and dried under vacuum. Yield 0.42 g.

### Example 10

### Calcium Risedronate

Risedronic acid (0.3 g) was dissolved in 2 mL of water and mixed with a solution of calcium hydroxide (0.037 g) in 40 mL of water. The mixture was stirred until completely clear and freeze dried to afford the product as a white powder. Yield 0.31 g.

### Example 11

### Magnesium Risedronate

Risedronic acid (0.3 g) was dissolved in 8 mL of water. Magnesium hydroxide (0.03 g) was added and the resulting mixture was stirred at room temperature until completely clear. The solution was concentrated to half volume and acetone was added to precipitate the salt. The product was filtered off, washed with acetone, and dried under vacuum to afford 0.31 g of the salt.

### EQUIVALENTS

Those skilled in the art will recognize, or be able to ascertain using no more than routine experimentation, numerous equivalents to the compounds and methods of use thereof described herein. Such equivalents are considered to be within the scope of this invention and are covered by the following claims.

All of the above-cited references and publications are hereby incorporated by reference.

1. A drug delivery device comprising a drug core comprising a low-solubility salt of a bisphosphonate.

2. The device of claim 1, wherein, upon administration, the drug delivery device provides sustained release of a therapeutically effective dose of the bisphosphonate for six to twelve months.

3. The device of claim 1, wherein the drug core is disposed in a tube having first and second ends, and the tube is sized and formed of a material so that the tube is dimensionally stable.

4. The device of claim 3, wherein the tube is permeable to passage of the bisphosphonate or the salt.

5. The device of claim 3, wherein the tube is impermeable to passage of the bisphosphonate and the salt, and at least one of the first or second ends is permeable to passage of the bisphosphonate or the salt.

6. The device of claim 5, wherein the tube further comprises a cap abutting the first and/or second ends of the tube, wherein the cap is permeable to passage of the bisphosphonate or the salt.

7. The device of claim 6, wherein the cap comprises at least one of (poly(vinyl acetate) (PVAC), poly(caprolactone) (PCL), polyethylene glycol (PEG), poly(dl-lactide-co-glycolide) (PLGA), ethylene vinyl acetate polymer (EVA), poly(vinyl alcohol) (PVA), poly(lactic acid) (PLA), poly(glycolic acid) (PGA), polyalkyl cyanoacrylate, polyurethane, nylon, or a copolymer thereof.

8. The device of claim 6, wherein the cap is bioerodible.

9. The device of claim 3, wherein the drug delivery device further comprises an outer layer that covers at least a portion of the tube, wherein the outer layer is permeable to passage of the bisphosphonate or the salt.

10. The device of claim 9, wherein the outer layer comprises at least one of poly(vinyl acetate) (PVAC), poly(caprolactone) (PCL), polyethylene glycol (PEG), poly(dl-lactide-co-glycolide) (PLGA), ethylene vinyl acetate polymer (EVA), poly(vinyl alcohol) (PVA), poly(lactic acid) (PLA), poly(glycolic acid) (PGA), polyalkyl cyanoacrylate, polyurethane, nylon, or a copolymer thereof.

11. The device of claim 9, wherein the outer layer is bioerodible.

12. The device of claims 3, wherein the tube comprises a polymer, a metal, or silicon.

13. The device of claim 12, wherein the tube is bioerodible.

14. The device of claim 13, wherein the tube comprises at least one of ethylene vinyl acetate polymer (EVA), polyalkyl cyanoacrylate, polyurethane, nylon, or a copolymer thereof.

15. The device of claim 1, wherein the bisphosphonate salt is a salt of a bisphosphonic acid with a divalent cation.

16. The device of claim 15, wherein the bisphosphonate is selected from alendronate, clodronate, etidronate, pamidronate, tiludronate, ibandronate, zoledronate, olpadronate, risedronate, incadronate, minodronate, and neridronate.

17. The device of claim 16, wherein the bisphosphonate is selected from ibandronate, zoledronate, and risedronate.

18. The device of any one of claims 15 to 17, wherein the divalent cation is a divalent metal cation selected from calcium, magnesium, manganese, and zinc.

19. The device of claim 15, wherein the bisphosphonate is ibandronate and the divalent cation is selected from calcium, zinc and benzathine.

20. The device of claim 15, wherein the bisphosphonate is zoledronate and the divalent cation is selected from magnesium, calcium and zinc.

21. The device of claim 15, wherein the bisphosphonate is risedronate and the divalent cation is selected from magnesium and calcium.

22. The device of claim 1, wherein the device comprises from about 10 mg to about 20 mg of the bisphosphonate.

23. A method for administering a bisphosphonate to a patient, comprising subcutaneously implanting the drug delivery device of claim 1.

24. The method of claim 23, wherein implanting the drug delivery device provides to the patient a therapeutically effective dose of a bisphosphonate for six to twelve months.

25. The method of claim 23, wherein said patient is suffering from osteoporosis, Paget's disease, or bone metastases.
